(19) United States
(12) Patent Application Publication
(10) Pub. No.: US 2016/0199232 A1
(43) Pub. Date: Jul. 14, 2016

(54) Heel Covering Device

(71) Applicant: Gail Stakes, Laramie, WY (US)

(72) Inventor: Gail Stakes, Laramie, WY (US)

(21) Appl. No.: 14/939,110

(22) Filed: Nov. 12, 2015

Related U.S. Application Data
(60) Provisional application No. 62/100,974, filed on Jan. 8, 2015.

(51) Int. Cl. A61F 13/06; A61F 13/00

(52) U.S. Cl. CPC A61F 13/064; A61F 13/00063; A61F 13/00085

(57) ABSTRACT

A heel covering device for applying ointment, lotion, moisturizer, or cream to a user's heel. The heel covering device forms a pocket into which a user's heel can be positioned, wherein the pocket substantially covers the user's heel when worn. The pocket is preferably composed of a thin plastic material so as to prevent any lotion placed in the pocket from escaping. The pocket further includes an absorbent edge about the perimeter so as to prevent ointment from escaping from the sides of the pocket. The heel covering device also includes a pair of straps extending from opposing sides of the pocket, wherein the straps are removably secured together over the front of a user's foot in order to secure the pocket onto the user's heel.

### CROSS REFERENCE TO RELATED APPLICATION

This application claims the benefit of U.S. Provisional Application No. 62/100,974 filed on Jan. 8, 2015. The above identified patent application is herein incorporated by reference in its entirety to provide continuity of disclosure.

### BACKGROUND OF THE INVENTION

### Field of the Invention

The present invention relates to heel covering devices. More specifically, the present invention provides a heel covering device for applying lotions, moisturizers, or other types of ointments to the user's heel while preventing the lotion from escaping the heel covering device.

Heels often become dry and cracked, requiring moisturizer, lotion, or other similar products to be applied to soothe and moisturize the person's heel. Further, ointments may need to be applied to a callous that develops on the heel. Without treatment, the dry and cracked skin on the heel can become painful and irritating. Thus, it is important to properly apply the necessary ointments.

However, applying an ointment directly to a person's heel can be problematic, as the ointment can easily rub off onto another surface, such as a sock, bedding, or a carpet. If the ointment rubs off onto another surface, the person will be unable to reap the benefits of the ointment, and the condition of the user's heel may not improve. Further, the ointment may stain or damage the surface with which it comes into contact. Thus, a device for facilitating application of ointments to a user's heel and retaining the ointment while it is applied is desired.

Devices have been disclosed in the prior art that relate to devices for treating feet. These include devices that have been patented and published in patent application publications. These devices generally relate to devices positioned on a portion of a user's foot for covering the foot or applying a moisturizer or ointment thereon, such as U.S. Published Patent Application Number 2005/0266059, U.S. Published Patent Application Number 2011/0308540, and U.S. Pat. No. 7,938,811. Other devices relate to coverings that enclose the user's foot or hand, such as U.S. Published Patent Application Number 2006/0064066, U.S. Published Patent Application Number 2014/0025024, and U.S. Published Patent Application Number 2013/0096515.

These prior art devices have several known drawbacks. For example, the devices in the prior art fail to provide a heel covering device that can be secured over a user's heel so as to apply lotions, ointments, or moisturizers to the user's heel. Other devices provide coverings for the entire foot, which can be inconvenient, and other devices relate to pads adhesively securable on the user. Adhesives may be unappealing to apply directly to the skin and the adhesives may not properly secure the pad to the user's foot or may leave a residue on the user's skin.

In light of the devices disclosed in the prior art, it is submitted that the present invention substantially diverges from and improves upon design elements from the prior art and consequently it is clear that there is a need in the art for an improvement to existing foot treatment devices. In this regard the instant invention substantially fulfills these needs.

### SUMMARY OF THE INVENTION

In view of the foregoing disadvantages inherent in the known types of heel covering devices now present in the prior art, the present invention provides a heel covering device comprising a pocket adapted to cover a user's heel, wherein the pocket is adapted to hold a cream or lotion therein for application to the user's heel. The pocket is composed of a thin, plastic material that contours to the user's heel and that prevents any lotion placed thereon from passing through the pocket. The perimeter of the pocket includes an absorbent edge for preventing the lotion from escaping from the sides of the pocket. The pocket further includes a pair of straps that can be tied or fastened around a user's foot in order to securely position the heel covering device on the user's heel without the heel covering device slipping or moving out of position.

### BRIEF DESCRIPTIONS OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a perspective view of an embodiment of the heel covering device.

FIG. 2 shows a side view of an embodiment of the heel covering device as positioned on a user's foot.

FIG. 3 shows a cross sectional view of an embodiment of the heel covering device.

### DETAILED DESCRIPTION OF THE INVENTION

Reference is made herein to the attached drawings. Like reference numerals are used throughout the drawings to depict like or similar elements of the heel covering device of the present invention. For the purposes of presenting a brief and clear description of the present invention, the preferred embodiment will be discussed as used for covering a user's heel so as to facilitate application of ointment on the user's heel. The figures are intended for representative purposes only and should not be considered to be limiting in any respect.

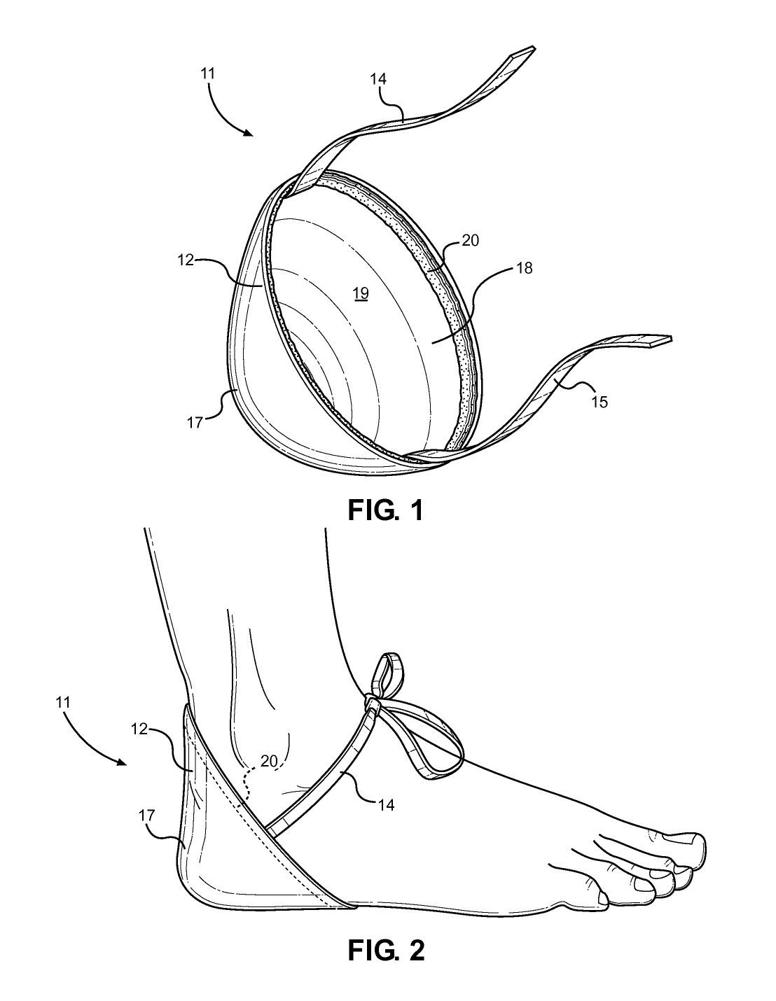

Referring now to FIGS. 1 and 2, there is shown a perspective view of an embodiment of the heel covering device and a side view of the heel covering device as positioned on a user's heel. The heel covering device 11 comprises a pocket 17 defining an interior volume 18 in which lotion or ointment can be deposited, and into which a user's heel can be positioned. A pair of straps 14, 15 extends from opposing sides of the pocket 17 for securing the heel covering device 11 on the user's heel. When in use, the pocket 17 extends from the bottom of the user's foot, around the heel, and up towards the user's ankle.

The pocket preferably has a rounded configuration so as to resemble a dome shape with a rounded or elliptical opening. The pocket 17 is preferably composed of a thin plastic or polymer that can contour to the shape of the user's heel. Further, the pocket 17 is constructed of a material that prevents any ointment or lotion placed on the inner surface 19 thereof from passing through to the exterior of the pocket 17. This helps to ensure that the ointment contacts the user's heel and prevents the ointment from rubbing off onto the user's clothing.

The perimeter 12 of the pocket 17 comprises an absorbent edge 20. The absorbent edge 20 helps to prevent lotion or ointment positioned on the inner surface 19 of the pocket 17 from escaping from the sides or edges of the pocket 17. The absorbent edge 19 is preferably composed of cotton or gauze, among other similar absorbent materials, so as to absorb and hold any lotion or cream that contacts the same. The absorbent edge 19 preferably extends around the entire perimeter of the pocket 17.

The pair of straps 14, 15 are used to removably secure the heel covering device 11 around the user's foot. A first strap 14 extends from a first a first side of the covering 17 and a second strap 15 extends from a second side of the covering 17, opposite the first side. The straps 14, 15 can be removably fastened around the front of the user's foot. Preferably, the user can manually tie the straps together in a knot. Alternatively, the straps may include hook and loop fastening material thereon for removably securing the straps together.

In other embodiments, the heel covering device 11 comprises an alternate fastener for securing the pocket 17 on the user's heel. In such embodiments, the fastener includes an elastic strap that extends between opposing sides of the pocket, such that the elastic strap can be stretched to fit over the user's foot.

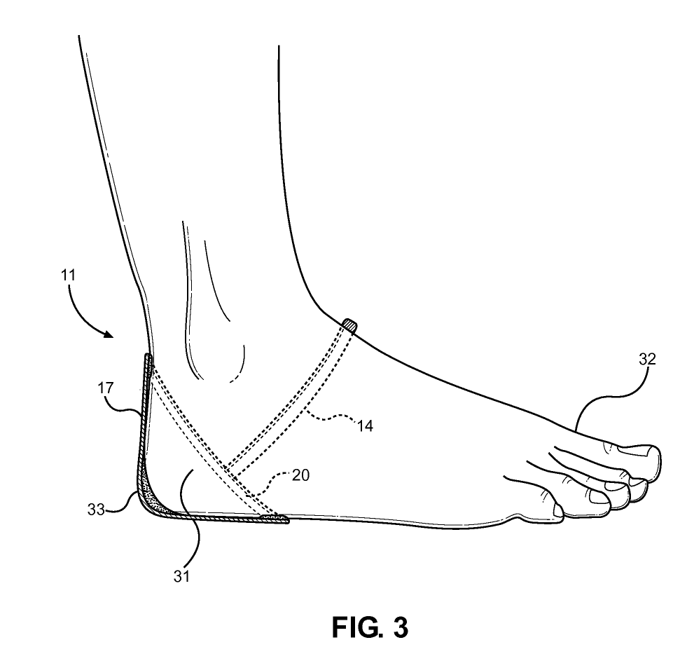

Referring now to FIG. 3, there is shown a cross sectional view of an embodiment of the heel covering device. In operation, the ointment 33 the user desires to apply to his or her heel 31 can be disposed on inner surface of the pocket 17. The pocket 17 can then be placed into contact with the user's heel 31 so that the pocket 17 covers the user's heel 31. The absorbent edge 20 on the perimeter of the pocket 17 prevents the ointment 33 from escaping from the sides of the pocket 17. Once the pocket 17 is in position, the user can fasten the straps 14 around the user's foot so as to securely position the heel covering device 11 on the user's heel so that the device 11 does not slip or move out of position throughout use thereof.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1) A heel covering device, comprising:
a pocket having a perimeter, wherein the pocket is adapted to cover a heel of a user;
an absorbent edge disposed on the perimeter of the pocket;
a first strap extending from a first side of the pocket, and a second strap extending from a second side of the pocket, wherein the first strap and the second strap are adapted to be removably fastened to one another so as to secure the pocket in position over the heel of the user.

2) The heel covering device of claim 1, wherein the first strap and the second strap are positioned on opposing sides of the pocket.

3) The heel covering device of claim 1, wherein the pocket is composed of a thin plastic material.

4) The heel covering device of claim 1, wherein the pocket comprises a dome shaped configuration.

5) The heel covering device of claim 1, wherein the absorbent edge is composed of cotton.

6) A heel covering device, comprising:
a pocket having a perimeter, wherein the pocket is adapted to cover a heel of a user;
an absorbent edge disposed on the perimeter of the pocket;
a fastener extending from the pocket and adapted to secure the pocket on the heel of the user.

7) The heel covering device of claim 6, wherein the fastener comprises an elastic strap.

8) The heel covering device of claim 6, wherein the fastener comprises a first strap having hook and loop fastening material thereon and a second strap having hook and loop fastening material thereon, wherein the first strap and the second strap can be removably engaged.
